(19) United States
(12) Patent Application Publication
(10) Pub. No.: US 2008/0318250 A1
(43) Pub. Date: Dec. 25, 2008

(54) Compositions and Methods for Identifying Sperm for Forensic Applications

(76) Inventors: Linda Gilmer, Palmyra, VA (US); Arabinda Mandal, Charlottesville, VA (US); Michael J. Wolkowicz, Charlottesville, VA (US); Kenneth L. Klotz, Schuyler, VA (US); John C. Herr, Charlottesville, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/588,445

(22) PCT Filed: Feb. 7, 2005
(86) PCT No.: PCT/US2005/003910

§ 371 Date: Aug. 4, 2006

Related U.S. Application Data
(60) Provisional application No. 60/542,499, filed on Feb. 6, 2004.
(60) Provisional application No. 60/581,945, filed on Jun. 22, 2004.

(51) Int. Cl. G01N 33/53; C07H 1/00; C12P 19/34

(52) U.S. Cl. 435/7.21; 536/25.41; 435/91.2

(57) ABSTRACT

Methods and compositions for identifying and isolating sperm cells from samples containing multiple cell types are described. The methods and compositions employ antibodies that specifically bind to sperm-specific antigens located on or internal to the sperm plasma membrane. A reporter molecule may be conjugated to the antibodies to aid in the detection of sperm. The antibodies may be targeted to sperm-specific antigens in the head and/or tail of sperm to facilitate the identification and isolation of sperm cells from forensic samples prepared from sexual assault evidence. Purified DNA from the isolated sperm cells can be amplified by polymerase chain reaction to assist forensic analysis in sexual assault cases.

### CROSS REFERENCE TO RELATED APPLICATIONS

This application is entitled to priority pursuant to 35 U.S.C. § 119(e) to U.S. provisional patent application Nos. 60/542,499, filed on Feb. 6, 2004, and 60/581,945, filed on Jun. 22, 2004.

### US GOVERNMENT RIGHTS

This invention was made with United States Government support under National Institutes of Health Grant Nos. T32 HD07382, T32 DK07642, and U54 29099, National Institute of Justice No. 2000-IJ-CX-K013, and Federal Bureau of Investigations No. 115744. The United States Government may therefore have certain rights in the invention.

### FIELD OF THE INVENTION

The present invention relates generally to detecting and isolating sperm in biological samples. More specifically, it is directed to the use of antibodies to identify, isolate, and purify sperm cells and sperm DNA from forensic samples in sexual assault cases.

### BACKGROUND

Sexual assault evidence recovered from a victim is an admixture of various cell types and fluids from both victim and assailant. In cases of vaginal assault, cells originating from the victim include cervical and vaginal epithelial cells, erythrocytes (red blood cells), white blood cells, various vaginal flora, including species of Lactobacillus, Candida, E. coli, as well as cervical mucus and minor contributions from uterine “milk.” Semen, the male component, contains roughly 85% seminal fluid originating from prostate and seminal vesicles, epithelial cells from these organs, spermatozoa, and epididymal fluid (15% of the ejaculate volume) and may contain white blood cells and various bacterial, viral or fungal commensals. In various cases of oral assault, buccal epithelial cells and buccal flora are often present as part of the female component. In cases of anal assault, a variety of intestinal and colonic epithelial cells, secretions, foodstuffs, and bacteria may be present in the victim's component.

Currently, the only stains available to aid in the identification of sperm are nuclear and cytoplasmic stains [such as the Christmas Tree stain] which are not specific for sperm but stain a variety of cells including vaginal and cervical epithelial cells, bacteria and cells sloughed from the male accessory sex glands. This leaves the positive identification of sperm relying on discovery of the characteristic shape and form of intact sperm, which may prove difficult as the sperm head and tail separate very easily after the sperm is dried and eluted from swabs. This problem of positive identification is particularly problematic where few numbers of sperm are present in the midst of a large number of other cells and debris. In such instances it may take a very long time for the forensic scientist to scan microscope slides in order to positively confirm the presence of sperm.

Once sperm are isolated from other components present in a forensic sample, PCR based analysis of sperm DNA can identify the source individual with a high degree of certainty. Many criminals have been identified after comparison of their DNA to the patterns recorded in the convicted offenders database (CODIS). Because of the great sensitivity of the PCR method it is possible to obtain useful data from a small number of recovered sperm, even as few a single sperm. However, with the advent of PCR based reactions and their increased sensitivity, the problem of defining the cellular source of amplified DNA and assigning, beyond a reasonable doubt, that source to the assailant, has proved a more difficult undertaking. A need for purer input DNA is thus inherent in the PCR protocol where all DNAs, including contaminating species, undergo amplification prior to analysis.

There is a long felt need in the art for a method to identify sperm in a field of debris and other cell types and to be able to isolate sperm from a field of debris and other cell types. The present invention satisfies these needs.

### SUMMARY OF THE INVENTION

One approach for identifying and isolating human sperm in a forensic sample comprises the use of ligands that specifically bind to unique sperm antigens. In one aspect, the antigens are sperm surface antigens. Such sperm specific antigens should be readily accessible for binding to a ligand (e.g. an antibody) and yet the sperm specific antigen must be sufficiently stable that the antigen is still present on the sperm, and capable of being recognized by the ligand, after recovery and storage of a forensic sample. As described herein applicants have discovered that sperm membrane antigens are often lost and are absent from sperm recovered from dried swabs prepared in sexual assault cases. This is because the plasma membrane is absent in many sperm eluted from post-coital swabs. Accordingly, the present invention is directed to a method of identifying and isolating sperm from a forensic sample. The method utilizes reagents that specifically bind to sperm specific compounds that are stable and persist on sperm heads and/or tails during the time and procedures used to recover forensic samples.

The present invention is directed to methods and compositions for identifying, isolating, and purifying sperm cells and sperm DNA from biological or forensic samples that comprise multiple cell types. In one embodiment, the method comprises selecting sperm cells based on sperm-specific antigens and separating them from other cell types. DNA can then be recovered from the isolated sperm cells and amplified by a polymerase chain reaction (PCR) using techniques known to those skilled in the art (See, e.g., Innis et al., Eds., 1990, in PCR Protocols, Academic Press, San Diego). In another embodiment, sperm-specific antibodies are used to isolate sperm cells before highly pure sperm DNA is isolated for subsequent PCR amplification. In a particular embodiment, the antibodies are monoclonal antibodies. In yet another embodiment, antibodies specific for antigens located on or internal to the sperm surface are bound to solid support (such as magnetic particles) to enhance cell separation and reduce the presence of contaminating cells in forensic evidence.

In one embodiment, the isolated sperm DNA is used for forensic DNA analysis of the “male component” in sexual assault evidence. In one aspect, the recovered sperm DNA is subjected to PCR analysis of short tandem repeat (STR) loci, providing an enabling technology to assist the development of the National Convicted Offender Database (CODIS). STR loci are simple tandemly repeated sequences of 1-6 base pairs (bp) in length which vary among individuals in the number of repeats exhibited. In another embodiment, the method for isolating sperm cells is automated in the form of a robotic device that interfaces with PCR probes for short tandem repeats. The method and device of the present invention improve the speed and accuracy of handling sexual assault evidence, thereby enhancing the development of CODIS.

One aspect of the invention for identifying and isolating human sperm in a forensic sample comprises the use of ligands that specifically bind to unique antigens located on or internal to the sperm surface. Such sperm-specific antigens should be readily accessible for binding to a ligand (e.g., an antibody). Moreover, the sperm-specific antigens must be sufficiently stable so that they are still present on or in sperm, and are capable of being recognized by the ligand, after recovery and storage of a forensic sample. As described herein, applicants have discovered that sperm membrane proteins are often lost and absent from sperm recovered from dried swabs prepared in sexual assault cases. This is because the plasma membrane is absent in many sperm eluted from post-coital swabs. Therefore, one aspect of the invention provides for the use also of ligands (e.g., antibodies) that specifically bind to sperm-specific antigens located internal to the sperm plasma membrane.

In yet another embodiment, a method of identifying and isolating sperm from forensic samples utilizes reagents, such as antibodies, that specifically bind to sperm-specific antigens that are stable and persist on or in sperm head and/or tail during the times and procedures used to recover, store, and handle forensic samples. In a particular embodiment, one or more antibodies directed against different sperm-specific antigens located on or in the sperm head and/or tail are used to rapidly detect sperm in smears from forensic samples. For example, a monoclonal antibody directed against a sperm-specific antigen would be the most selective reagent to use for sperm immunoselection. Accordingly, one aspect of the invention provides for the use of sperm-specific monoclonal antibodies. Sperm-specific protein antigens located in the sperm head and/or tail include, but are not limited to, SP-10, CABYR, CBP86, ESP, SAMP14, SAMP32, SPAN-X, and AKAP.

In one aspect of the invention for rapidly detecting sperm in biological or forensic samples, sperm-specific antibodies targeted to epitopes on or in the sperm head and/or tail are either directly or indirectly conjugated to a reporter molecule such as a dye or a fluorescent label. In one embodiment the antibodies are directly conjugated to the reporter molecule, and in another embodiment the reporter molecule is conjugated to a secondary antibody that recognizes the primary sperm-specific antibody. If a fluorochrome is utilized, antibody-bound sperm heads and/or tails are easily identifiable under fluorescent microscopy, even if the heads and tails have separated, as they fluoresce brightly against a negative background.

One embodiment of the invention provides for an automated method and device for identifying, isolating, and purifying sperm cells and sperm DNA from biological or forensic samples. Sperm cells are isolated by using a mixture of different types of magnetic beads or particles, each bead type being coated with a different antibody specific for a different sperm-specific antigen, at least one antibody being targeted to an antigen located internal to the plasma membrane in the sperm head. Methods for attaching or conjugating antibodies to other entities or to a solid support such as chromatographic media and magnetic particles are known in the art. At least one antibody to an antigen located on the sperm plasma membrane and at least one antibody to an antigen located internal to the plasma membrane in the sperm tail may also be coupled to the magnetic beads. The antibodies may also be either directly or indirectly conjugated to a reporter molecule such as a dye or fluorescent marker. After the sample is incubated with the magnetic beads in a vessel, any sperm cells present will bind to the antibody-coated magnetic beads. A magnetized probe or a robotic arm coupled to an electromagnet picks up the sperm-bound magnetic beads, and the beads are washed to remove any unbound or non-specifically bound material. Next, the probe is contacted with a cell lysis solution in another vessel, where the sperm cells are lysed. The probe is then removed from the vessel and sperm DNA is isolated and purified, using standard techniques, for subsequent PCR amplification.

Various aspects and embodiments of the invention are described in further detail below.

### BRIEF DESCRIPTION OF THE DRAWINGS

FIG. 1 demonstrates a fluorescent microscopy image illustrating that the 3C6 antibody against ESP can be used to identify sperm heads in a post-coital sample eluted from a cotton swab.

FIG. 2 demonstrates a fluorescent microscopy image illustrating that the 3A4 antibody against CABYR can be used to identity sperm tails in a post-coital sample eluted from a cotton swab.

### DETAILED DESCRIPTION OF EMBODIMENTS

### Definitions

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

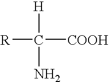

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino-and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The terms “detect” and “identify” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator “http://www.ncbi.nlm.nih.gov/BLAST/”. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used. See http://www.ncbi.nlm.nih.gov.

The percent identity between two sequences can be determined using techniques similar to those described above, with or without allowing gaps. In calculating percent identity, typically exact matches are counted.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “secondary antibody” refers to an antibody that binds to the constant region of another antibody (the primary antibody).

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

As used herein, the term “magnetic particles” refers to particles that are responsive to a magnetic field.

“Sperm-specific”, as used herein, refers to an antigen which is present at higher levels on sperm than other cells or is exclusively present in sperm.

A “test sample”, as used herein, refers to a sample of semen or to a sample obtained as a forensic sample such as a post-coital swab.

Used interchangeably herein are the following pairs of words (1) “detect” and “identify”; (2) “select” and “isolate”; and (3) “sperm surface” and “sperm plasma membrane.”

As used herein, the term “SP-10 antibody” and like terms refer to an antibody or fragment thereof that specifically binds to a polypeptide comprising SEQ ID NO:1 or a fragment of SEQ ID NO:1.

As used herein, the term “CABYR antibody” and like terms refers to an antibody or fragment thereof that specifically binds to a polypeptide comprising SEQ ID NO:2 or a fragment of SEQ ID NO:2.

As used herein, the term “ESP antibody” and like terms refers to an antibody or fragment thereof that specifically binds to a polypeptide comprising SEQ ID NO:3 or a fragment of SEQ ID NO:3.

As used herein, the term “SAMP32 antibody” and like terms refers to an antibody or fragment thereof that specifically binds to a polypeptide comprising SEQ ID NO:4 or a fragment of SEQ ID NO:4.

As used herein, the term “SPAN-X antibody” and like terms refers to an antibody or fragment thereof that specifically binds to a polypeptide comprising SEQ ID NO:5 or a fragment of SEQ ID NO: 5 (see International Application PCT/US99/24973 (GenBank accession number AAF28420.1), the disclosure of which is incorporated in its entirety herein), or other members of the SPAN-X family. As used herein, SPAN-X includes the SPAN-X proteins such as SPAN-Xa and SPAN-Xb.

As used herein, the term “AKAP antibody” and like terms refers to an antibody or fragment thereof that specifically binds to a polypeptide comprising SEQ ID NO:6 or a fragment of SEQ ID NO:6 as provided by GenBank accession number AF087003.

### Various Embodiments of the Invention

The present invention is directed to methods and compositions for identifying, isolating, and purifying sperm cells and sperm DNA from biological or forensic samples that comprise multiple cell types. One embodiment is directed to methods and compositions for rapidly identifying human sperm in sexual assault evidence. In one aspect of the invention, protocols are designed for rapidly determining the presence of sperm in a sample when the number of sperm is low, e.g., in a sample eluted from sexual assault swabs. Reduction in the amount of time required to positively identify human sperm in sexual assault samples is anticipated to provide a cost saving in forensic practice as well as expedite the number of cases processed, particularly in situations where sperm is mixed with a variety of other cells and unknown material. In another aspect of the invention, compositions are provided for rapidly isolating and purifying sperm from sexual assault evidence to allow recovery and analysis of sperm DNA.

In one aspect of the invention, antibodies directed against sperm-specific antigens are used to identify and isolate sperm cells from complex biological mixtures. For an antigen to be useful in sperm immunoselection, it must be present and accessible on or in sperm and must not react with other cell types, including vaginal epithelial cells that may be present in the biological sample. The selection of the sperm-specific antigen is particularly important for samples that are recovered from dried swabs or forensic evidence, since the sperm plasma membrane is frequently lost during the recovery of such samples. In one embodiment, the sperm-specific antibodies are targeted to sperm surface antigens (present on the surface of either acrosome-reacted or non-reacted sperm cells). In another embodiment, the selected sperm-specific target molecules are restricted to the sperm head and/or tail so that where the head separates from the tail, each can be positively identified.

In another aspect of the invention, sperm-specific antibodies are used in post-coital testing. In one embodiment, the sperm-specific antibodies are used to detect and/or quantitate the presence of sperm in cervical mucus. Such detection can be used to determine whether individuals have engaged in sexual intercourse and can be used in forensic analysis.

Several sperm-specific proteins have been previously described (See, e.g., U.S. Pat. No. 5,436,157 (SP-10) and international patent application nos. PCT/US99/24973 (Span-X), PCT/US01/01715 (CBP86), PCT/US00/02675 (AKAP), and PCT/US01/01717 (ESP & SAMP32), all of which are incorporated by reference herein in their entirety) that have the potential to permit the rapid detection of sperm in smears from forensic samples. However, as reported in Example 2 of the present invention, sperm-specific antigens that are located on the plasma membrane may not be retained on sperm that have been subjected to standard forensic recovery, storage, and handling procedures. Accordingly, in one embodiment of the invention, an effective method and composition for identifying sperm in a complex biological mixture is based on targets that persist in sperm and can be detected when swabs are collected and allowed to dry before analysis of the recovered sample. In another embodiment, the sperm-specific target compounds are selected from those that persist in sperm for extended time periods up to 72 hr after sexual intercourse.

As reported herein (See Example 2), the ESP, SPAN-X, CABYR, SP-10, and SAMP32 proteins are present and can be detected in many sperm when swabs are collected one to two hours after sexual intercourse. In this experiment, sperm was stained with a sperm-specific antibody to which was bound a fluorescently conjugated secondary antibody. In addition, the sperm-specific tail protein AKAP3 can be detected in sperm recovered from swabs and stains with a very bright fluorescent signal over the principal piece of the tail. AKAP3 is a very abundant sperm tail protein that appears to persist for some time in the sperm tail after sexual intercourse. The fibrous sheath proteins CABYR and AKAP3 are expected to be the most resilient and to be detectable for the longest period of time after sexual intercourse. Other useful sperm antigens include, but are not limited to, SAMP14 (acrosome), CBP86 (tail), HUP1N (condensed sperm nucleus) and HUP2B (condensed sperm nucleus). Other sperm antigens, either known or not yet known, are also contemplated for use in the invention described herein.

One embodiment of the invention provides for a composition for labeling sperm head and/or tail. A particular embodiment provides for a composition that specifically binds to post-coital sperm cells. In one embodiment, the composition comprises an antibody that binds to a polypeptide, or to a fragment thereof, selected from the group of polypeptides consisting of SEQ ID NO:1 (SP-10), SEQ ID NO:2 (CABYR), SEQ ID NO:3 (ESP), SEQ ID NO:4 (SAMP32), SEQ ID NO:5 (SPAN-X), and SEQ ID NO:6 (AKAP3). In a particular embodiment, the antibody is a monoclonal antibody, and in another particular embodiment, it is a polyclonal antibody. In another embodiment, the sperm-labeling composition comprises a cocktail of two or more antibodies, each antibody being monoclonal or polyclonal. In a further embodiment, the composition comprises two or more sperm-specific antibodies, each antibody staining a different sperm head or tail antigen. In a more particular embodiment, the composition comprises at least one antibody that binds to a sperm head-specific antigen and at least one antibody that binds to a sperm tail-specific antigen. In yet another embodiment, the composition comprises an antibody that binds to a sperm-specific antigen that is located on or in both the head and the tail of sperm and is retained at least two hours after ejaculation. Other antibodies not described herein, either known or not yet known, directed against other sperm antigens either described or not described herein, either known or net yet known, are also contemplated for use in the invention described herein.

In another embodiment, protamines (including protamine 1 (HUP1N) and protamine 2 (HUP2B) are selected as target compounds in sperm heads recovered from swabs. The major form of most sperm cells recovered from dried cotton swabs is a condensed nucleus with few membranes attached. Protamines are very abundant found only in the nucleus of sperm. Applicants have determined conditions that will cause partial decondensation of dried sperm nuclei recovered from swabs, exposing protamine epitopes, and that may be adopted in forensic laboratories, enabling the use of anti-protamine antibodies for sperm immunoselection.

A cocktail of sperm-specific antibodies used to detect or isolate sperm may comprise a sperm-specific antibody targeted to a sperm surface antigen. One such sperm surface antigen is SAGA-1, which is a unique sperm surface carbohydrate epitope—sperm agglutination antigen-1. This antigen is synthesized in the principal cells of the epididymis, is specific to the male reproductive tract of humans and higher primates, and is inserted by way of a glycophosphotidylinositol (GPI) anchor into all domains of the sperm surface—the head and the midpiece, principal piece, and end piece of the tail of sperm.

The antibodies of the present invention can be combined with a carrier or diluent to form a composition. In one embodiment, the carrier is a pharmaceutically acceptable carrier. In another embodiment, the antibodies are linked to a solid support. In yet another embodiment, the antibodies are linked to a detectable marker.

The methods of identifying or isolating sperm cells using sperm-specific antibodies can employ a variety of detectable markers, or reporter molecules, that are either directly linked or indirectly linked to the sperm-specific antibodies. Such detectable markers or reporter molecules include, but are not limited to, colorimetric molecules, fluorescent molecules, chemiluminescent molecules, or horseradish peroxidase (HRP). If a plurality of sperm-specific antibodies are employed to detect or isolate sperm cells, all the antibodies may be directly or indirectly conjugated to the same reporter molecule, or each of the antibodies may be directly or indirectly conjugated to a different reporter molecule.

Under suitable conditions, a calorimetric reporter molecule forms a color or changes color, a fluorescent reporter molecule fluoresces or changes fluorescence, and a chemiluminescent reporter molecule chemiluminesces, or emits light due to a chemical reaction. Horseradish peroxidase (HRP) may be considered to be a colorimetric reporter molecule. An antibody-IRP conjugate causes precipitation of a colored substrate where the antibody binds to the corresponding antigen.

A reporter molecule may be an enzyme or an enzyme substrate. If the reporter molecule is an enzyme, the corresponding enzyme substrate is added after the antibody is allowed to bind to the corresponding antigen. If the reporter molecule is an enzyme substrate, the corresponding enzyme is added. Reaction between the enzyme and the enzyme substrate gives rise to the formation of a color, a change in color, fluorescence, a change in fluorescence, or chemiluminescence.

In one embodiment, the antibodies are labeled either directly or indirectly, using an immunofluorescence compound and techniques known to those skilled in the art. In the direct method, the antibodies are labeled directly with a fluorochrome. In the indirect method, the fluorochrome is attached to a secondary antibody that recognizes the sperm-specific antibody. In one embodiment, the sperm-specific antibodies are monoclonal antibodies that have been directly conjugated to a fluorochrome. Using fluorescence microscopy, the equatorial band signal for a positive head or a fluorescing sperm tail is very strong and easily identifiable at 400×, even if the head and tail have separated.

The indirect method has the advantage that it can amplify the fluorescent signal by binding more fluorochrome at the antigen site. Therefore, its potential fluorescent signal on sperm may be stronger than the direct method, especially at low antibody-conjugate concentrations. A drawback of the indirect method is that it employs two separate steps of antibody addition.

The direct method has the advantage that it reduces the number of washing steps and is quicker. The use of a single labeled immunoreagent also reduces the background fluorescence by eliminating non-specific binding of the secondary antibody. One possible drawback of using a single labeled immunoreagent is that at low antibody-antigen ratios, the fluorescent signal may be lower than that in the indirect method.

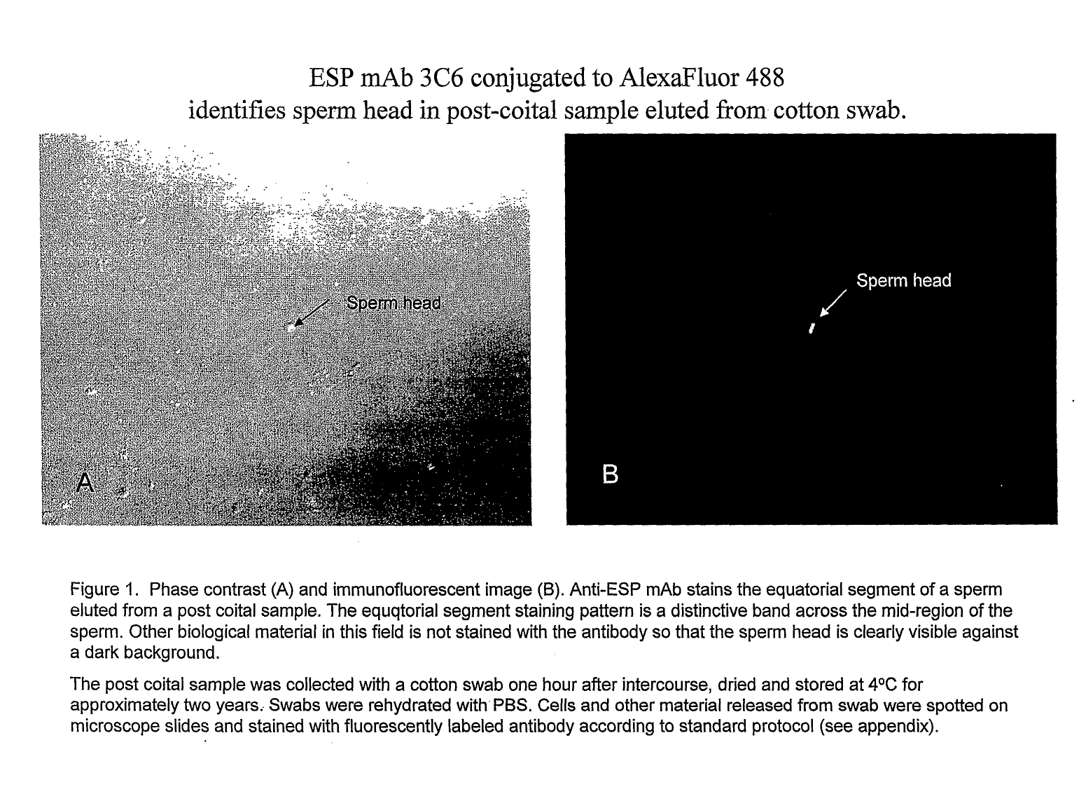

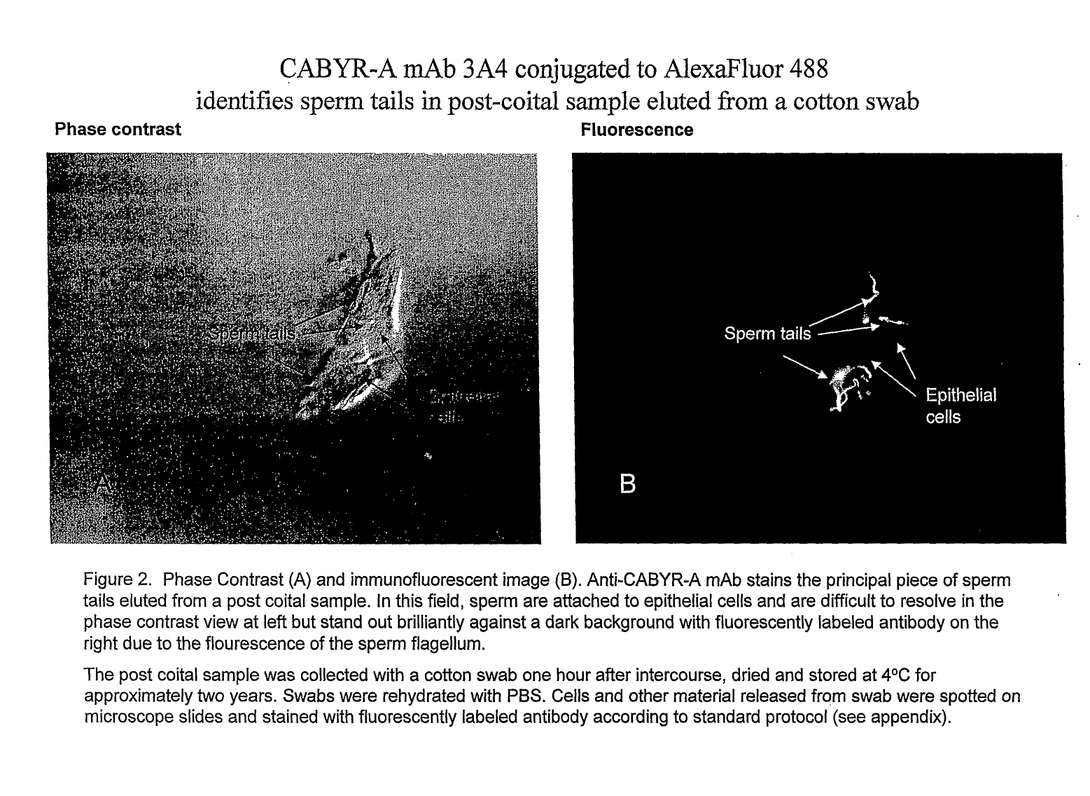

Fluorescently labeled sperm-specific antibodies are very effective reagents for unequivocally identifying sperm in forensic samples. In tests, as illustrated in FIG. 1 and FIG. 2, the fluorescent signal was bright and sperm were easily distinguished from the background and other contaminating cell types and debris. Until every forensic lab has access to a fluorescent microscope, however, an alternative approach using sperm-specific antibodies would be of great value.

Accordingly, one aspect of the present invention is directed to the use of horseradish peroxidase (HRP) conjugates of sperm-specific antibodies to immunostain sperm in forensic samples. HRP conjugates stain cells by causing precipitation of a colored substrate where the antibody is bound to the cell. Other commercially available reporter molecules or substrates include, e.g., True Blue® (tetramethyl benzidine, TMB) from KPL Laboratories and NovaRED® from Vector Laboratories.

In an aspect of the invention, a composition for labeling sperm cells comprises an antibody specific for the equatorial segment protein (ESP) protein (SEQ ID NO: 3) and an antibody specific for a protein selected from the group consisting of AKAP3 (SEQ ID NO: 6) and CABYR (SEQ ID NO: 2). The ESP protein represents an epitope in the sperm head, whereas the AKAP3 and CABYR proteins represent epitopes in the sperm tail. In one embodiment, the antibodies are monoclonal antibodies.

The 3C6 monoclonal antibody binds to ESP and stains the equatorial segment of sperm heads (See FIG. 1). The 3A4 monoclonal antibody binds to the calcium binding tyrosine phosphorylated protein (CABYR-A) and stains the principal segment of sperm tails (See FIG. 2). The 3A5 monoclonal antibody also binds to CABYR-A. Other useful sperm proteins include SAMP14 (acrosome), SAMP32, SP-10, SPAN-X, and CBP86. Both antibodies stain sperm present in post-coital evidence which has been stored for up to two years. When used in immunofluorescent microscopy using FITC-conjugated secondary antibodies, the sperm are easily identified as they fluoresce brightly against a negative background. The sperm head-staining monoclonal antibody 3C6 gives a characteristic band across the mid-region of the head that corresponds to the domain of the equatorial segment. The 3A4 monoclonal antibody stains the principal segment of the tail most intensely. Other useful antibodies for identifying sperm include, but are not limited to, mAb A9, directed against SPAN-X, rat antisera to SAMP32, polyclonal antisera against CABYR, 3A5 against CABYR, MHS-10 mAb against SP-10, and mAb 8G8G8G8 against SAMP14.

Another aspect of the invention provides for a method of rapidly detecting the presence of human sperm in a biological sample, including sperm recovered from dried stains on clothing, from vaginal swabs, from material collected by lavage with physiological saline, and from any suspension which includes sperm. The method uses a reporter molecule-labeled antibody which specifically binds to a human sperm-specific antigen that is retained and accessible to an antibody after the sperm-containing specimen has been dried and subsequently rehydrated, and comprises the steps of contacting the sample with the labeled antibody and detecting for the presence of the labeled antibody. In another embodiment, the method further comprises the step of removing unbound and non-specifically bound material to purify one or more sperm cells from the sample. In a particular embodiment, the antibody used in the method of identifying and/or isolating sperm is an antibody that specifically binds to a sperm-specific protein comprising a sequence selected from the group consisting of SEQ ID NO: 1, SEQ ID NO: 2, SEQ ID NO: 3, SEQ ID NO: 4, SEQ ID NO: 5, and SEQ ID NO: 6 or fragments thereof.

In one aspect of the invention, the methods using antibodies to label sperm cell components are especially useful when the sperm head and tail have become separated, and the shape and form of the sperm under light microscopy are difficult to discern. Using fluorescent microscopy, the equatorial band signal for a positive head or a fluorescing sperm tail is very strong and easily identifiable, even if the head and tail have separated. Considering that sperm membrane antigens are often lost and absent from sperm recovered from dried swabs in sexual assault cases, one embodiment of the present invention employs antibodies that are specific for sperm-specific antigens that are retained on or in sperm eluted from dried post-coital swabs. In another embodiment, the target antigens are selected from those that are retained on or in sperm eluted from dried post-coital swabs which have been stored for greater than 72 hours, and even up to two years.

Another aspect of the invention is directed to a method of purifying sperm DNA from a biological sample that comprises multiple cell types. The method comprises selecting sperm cells and separating them from other cell types using the sperm cell-selection methods described above, recovering DNA from the selected sperm cells, and amplifying the recovered sperm DNA by a PCR reaction using techniques known to those skilled in the art. In one embodiment, sperm-specific antibodies are used to isolate highly pure sperm DNA for subsequent PCR amplification.

In yet another aspect of the invention, sperm heads and/or tails are isolated from a biological sample using antibodies that target sperm-specific antigens located internal to the sperm plasma membrane. In a particular embodiment, the sperm-specific antigens include SP-10, ESP, SPAN-X, SAMP14, CBP86, SAMP32, AKAP3, HUP1N, HUP2B, and CABYR. Since sperm cells differ in the extent of the loss of their plasma membrane depending on the source and age of the sample as well as the procedures used to recover, store, and handle the sperm, in one aspect of the invention a cocktail of sperm-specific antibodies is employed. In one embodiment, the sperm-selecting composition comprises two or more antibodies that specifically bind to different sperm-specific epitopes located on different layers of and/or internal to the sperm plasma membrane.

In another embodiment, the sperm immunoselection cocktail comprises sperm-specific antibodies that bind to a broad range of sperm cells varying in the amount of retained plasma membrane. In one embodiment, the antibodies are monoclonal antibodies. In a particular embodiment, the cocktail includes antibodies directed against the sperm-specific proteins SP-10, ESP, and SPAN-X. In another particular embodiment, the cocktail comprises antibodies directed against the sperm-specific proteins CABYR, SP-10, ESP, and SPAN-X. In yet another embodiment, the cocktail of antibodies specific for multiple unique sperm antigens comprises at least two, and more preferably three, antibodies selected from the group consisting of the AKAP3 antibody, SP-10 antibody, ESP antibody, SAMP32 antibody, CABYR antibody, and SPAN-X antibody. In still another embodiment, the cocktail further comprises an antibody directed against protamine 1 and protamine 2. Protamine is an extremely abundant protein found only in the sperm nucleus and may prove to be an effective target for de-membranated sperm heads recovered, e.g., from swabs.

Sperm-Specific Antibodies Bound to Solid Support

Antibodies specific for sperm-specific antigens located on or internal to the sperm plasma membrane can be bound to solid support (such as magnetic particles) to enhance cell separation and reduce the presence of contaminating cells in forensic evidence, e.g., as described in U.S. patent application Ser. No. 10/146,552, which is incorporated by reference herein in its entirety. Non-limiting examples of sperm-specific antibodies are MHS 10, which recognizes the sperm acrosomal protein SP-10, and antibodies to SPAN-X, a sperm protein present in nuclear vacuoles and sperm nuclear redundant membranes.

In one embodiment, a biological or forensic sample containing sperm cells is contacted with a binding substrate comprising a solid support and an antibody directed against a sperm-specific antigen located on or internal to the sperm plasma membrane, where the antibody is linked to the solid support. The sample is incubated with the binding substrate for an amount of time sufficient to allow sperm cells to bind to the binding substrate. The binding substrate is then washed with a buffered solution to remove any unbound and non-specifically bound material. The sperm cells bound to the binding substrate are then lysed and sperm DNA is recovered and purified using standard techniques. In another embodiment, the solid support contains a plurality of different antibodies linked to it, where each antibody specifically binds to a different sperm-specific antigen located on or internal to the sperm plasma membrane.

Sperm-specific antibodies can be bound to solid support using techniques known to those skilled in the art. For example, the antibodies can be directly linked to functional groups at the surface of the solid support or can be attached to the solid support via a linker moiety. The linkage is preferably a covalent bond, although other linkages are also acceptable. In one embodiment, the sperm-specific antibodies are linked to the solid support via an antibody linker, where the linker is a secondary antibody that binds to the constant region of the sperm-specific primary antibody. In another embodiment, the linker is an enzymatically cleavable or photolytic linker. Linkers suitable for use in accordance with the present invention are well known to those skilled in the art.

In one embodiment, the solid support comprises a single solid surface. In another embodiment, the solid support is in particulate form. The particles may vary in shape and can be, e.g., round, rectangular, or irregularly shaped. Irregular shape adds more surface area, increasing the particles' binding capacity compared to larger spherical particles. The particles may also vary in size. Smaller particles may be more diffused throughout the sample solution, increasing target capture rate while decreasing incubation time. The size of the particles is important in limiting shear forces during the recovery of sperm cells. Preferably, the size of the particles is less than 4 μm, more preferably from about 10 nm to about 1 μm, and even more preferably from about 50 nm to about 500 nm. In a particular embodiment, the size of the particles ranges from about 100 nm to about 300 nm. In one embodiment, the solid support particles, to which are bound sperm-specific antibodies, are combined to form a column, and the biological or forensic sample is run through the column, followed by repeated washings, to isolate sperm cells.

In one aspect of the invention, the solid support comprises magnetic beads or particles linked to sperm-specific antibodies. In a particular embodiment, the antibodies are monoclonal antibodies. In one embodiment, the magnetic beads or particles are each coated with one or more different antibodies specific for different sperm-specific antigens. In another embodiment, a mixture of different types of magnetic beads or particles is used, each bead type being coated with a different antibody specific for a different sperm-specific antigen. The use of a bead with more than one type of antibody directed against different sperm-specific antigens, or a mixture of different bead types coated with different antibodies directed against different sperm-specific antigens is anticipated to result in binding to a higher proportion of sperm and the isolation of a higher percentage of enriched sperm than if only one bead type coated with only one antibody is used.

As described above, the plasma membrane is often lost or absent from the surface of sperm recovered from dried swabs. Therefore, antigens located on the plasma membrane, such as SAGA-1, may not be the best targets for sperm immunoselection when the plasma membrane is no longer associated with the rest of the sperm, particularly the nucleus. Nevertheless, sperm eluted from swabs that do retain fragments of the plasma membrane may effectively be bound by magnetic beads or particles coated w/an antibody specific for a sperm surface antigen, such as the S19 mAb which is specific for SAGA-1.

One embodiment of magnetic immunoselection employs a mixture of different types of magnetic beads or particles, each bead type being coated w/a different antibody to a different sperm-specific antigen, including at least one antigen located on the sperm plasma membrane and at least one antigen located internal to the plasma membrane. In one aspect of the invention, the sperm-specific antigens selected are those that are exposed and retained on dried sperm recovered from sexual assault swabs. In another aspect, the sperm-specific antigens selected are those that are located in subcellular compartments of sperm cells such as the nucleus, mitochondrial sheath, and fibrous sheath. In yet another aspect, the sperm-specific antigens selected are those that are located in structural elements and compartments unique to the sperm head, including the inner and outer acrosomal membranes, acrosomal matrix, subacrosomal cement (perinuclear theca), and nucleus. Representative, non-limiting examples of antigens unique to the sperm head are protamines, transition proteins of the nuclear matrix, and unique proteins of the nuclear envelope.

The magnetic beads or particles may be selected from among different types of magnetic beads and particles that are commercially available. Examples of paramagnetic beads include Miltenyi Biotech 50 nm dextran-coated microbeads and Micromod Nanomag-D and Nanomag-D-CO₂H beads. Smaller magnetic particles may move more slowly toward a magnetic source so that there would be less shear force to dislodge captured sperm, compared to larger beads.

When the solid support comprises magnetic particles, sperm cells can be easily separated from contaminates in the sample and from wash solutions by applying a magnetic field. In one embodiment, after sperm cells have bonded to the antibody-bearing magnetic particles, a source of magnetism can be applied to an exterior surface of the vessel containing the biological or forensic sample. The magnetic force immobilizes the sperm-bound magnetic particles on the interior surface of the vessel, allowing the remaining contents to be removed, e.g., by aspiration. The magnetic force can be continuously applied during the washing steps and while the sperm cells are being lysed. In one embodiment, after the last wash has been removed from the sample vessel, the magnetic force is deactivated and the magnetic particles with sperm cells attached to them are resuspended in buffer, and then the sperm cells are lysed.

### Device Embodiments of the Invention

The use of magnetic particles, to which are linked sperm-specific antibodies, allows the methods of isolating sperm cells and purifying sperm DNA from biological or forensic samples to be automated. The aforementioned U.S. patent application Ser. No. 10/146,552 describes various methods, means, and devices for automation. In particular, robotic arms can be used to add, remove, or transfer fluids from one vessel container to another, and robotic arms coupled to electromagnets can be employed to move sperm-bound magnetic particles w/in a vessel and between vessels. The computer software and the mechanical hardware necessary for conducting such automation are known to those skilled in the art and are described in, e.g., U.S. Pat. Nos. 5,366,896 and 5,128,103, which are incorporated by reference herein in their entirety.

In one embodiment, a method and device is provided for isolating sperm cells and purifying sperm DNA from a sample comprising sperm cells and other cell types. The sperm cells bind to one or more different antibodies specific for different sperm-specific antigens located on or internal to the sperm plasma membrane, where the antibodies are linked to magnetic particles. A robotic arm coupled to an electromagnet is programmed to place the electromagnet into a first compartment in which the antibody-linked magnetic particles are incubated with the sample. The electromagnet picks up sperm-bound magnetic particles, and the magnetic particles are washed to remove any unbound or non-specifically bound material. The robotic arm then transfers the electromagnet to a second compartment where the sperm cells are lysed. Sperm nucleic acid can then be isolated and amplified by techniques known in the art. In another embodiment, the device further comprises a metallic pin magnetically coupled to the electromagnet. In yet another embodiment, the device comprises a magnetic probe that is used to move the magnetic particles within a compartment and between compartments. In still another embodiment, the device can further be provided with a second magnetic source (either a fixed magnet or an electromagnet) located outside the second compartment but in close enough proximity to the second compartment so as to impart a magnetic force on the contents of the second compartment. The second magnet is used to assist in removing the magnetic particles from the first electromagnet after it is deactivated.

The device can further be provided with automated means for dispensing liquid into and withdrawing liquid from the various compartments. In one embodiment, the automated dispensing and withdrawing means comprise a system of positive and negative pressure pumps that direct fluids through tubes to the various compartments. In another embodiment, the automated dispensing and withdrawing means comprise one or more dispensing tubes attached to separate robotic arms, where the dispensing and withdrawal of fluids to/from specific compartments are programmed.

In another aspect of the invention, an immunochromatographic device is used to detect trace amounts of sperm in forensic samples. The device, possibly called SpermCheck Forensics, is utilized as a first line of testing to detect the presence of sperm in forensic samples. The device does not require a microscope evaluation and gives a Yes/No answer within five minutes of applying a suspension of cells eluted from a sample. The presence of sperm is identified by their binding to a first sperm-specific antibody, which is linked to a detectable marker, and binding to a second sperm-specific antibody, which is linked to a solid surface. Chromatographic means are employed to move a loaded sample to a target area of the solid surface where the second antibody is located and captures sperm labeled by the first antibody. The first and second antibodies are selected from those that specifically bind to different sperm-specific antigens located internal to the sperm plasma membrane. In a particular embodiment, the target sperm antigens are selected from the group consisting of SEQ ID NO: 1, SEQ ID NO: 2, SEQ ID NO: 3, SEQ ID NO: 4, SEQ ID NO: 5, and SEQ ID NO: 6.

The encouraging results from using prototypes of SpermCheck Vasectomy to detect sperm in post-coital samples eluted from cotton swabs (See Example 4) indicate that the prototypes can be optimized to produce a more sensitive device for detecting sperm in forensic samples. The more sensitive SpermCheck Forensics device will also be tested on post-coital samples collected up to 72 hours after intercourse. Any non-specific background reaction will have to be carefully controlled to ensure that the device can detect low levels of sperm without producing false positives in samples that contain no sperm. If the device can accurately test for the presence of sperm in a short period of time (e.g., five minutes), it would be greatly useful in quickly detecting sperm in forensic samples without the need for a microscope and a laboratory setting.

Assays for the Detection of Sperm

A biological or forensic sample is added to a preparation including one or more antibodies specific for one or more different sperm-specific antigens. The antibodies may be polyclonal or monoclonal, preferably monoclonal. The presence of sperm in the sample gives rise to binding between the particular antibody and the sperm antigen for which the antibody is specific, a binding reaction which is detected, directly or indirectly, through a variety of methodologies, e.g., those described in U.S. Pat. No. 5,605,803, which is incorporated by reference herein in its entirety.

A familiar type of assay is a calorimetric assay, in which a sperm-specific antibody is labeled with a reporter molecule which can be detected by a specific color. A binding reaction between the antibody and the corresponding sperm-specific antigen induces the formation of a color or a color change, or the color is developed with a second agent, typically an enzyme. As the reporter molecule is “developed” (i.e., the appropriate color is induced), only in the presence of sperm-bound antibody, a “positive” reaction is indicative of the presence of sperm, as the antigen is specific to sperm. The absence of the desired color (or the presence of a different color) is indicative of a “negative” result, i.e., an absence of the sperm-specific antigen, and therefore sperm, from the sample.

Among the easiest assays of this type to perform are solid-phase immunoassays, in which a first sperm-specific antibody, preferably a monoclonal antibody, is bound to a solid surface, such as a membrane or bed, which is exposed to a sample. Any sperm present in the sample binds to the first antibody. Any unbound or non-specifically bound material is washed or removed from the solid surface, followed by the addition of a second antibody which binds to a sperm-specific antigen and bears a reporter molecule or a label such as an enzyme or enzyme substrate. The second antibody need not bind to the same sperm epitope as the first antibody. After binding to the second antibody is allowed to occur, the solid surface is washed to remove any unbound or non-specifically bound material. Conditions are then established so that the reporter molecule or label may give a readily detectable signal which is indicative of the presence of sperm.

If the second antibody is labeled with an enzyme or enzyme substrate, the counterpart of the enzyme or enzyme substrate is added after washing the solid surface. (When the second antibody is bound to an enzyme, the enzyme substrate is added. When the second antibody is bound to the enzyme substrate, the enzyme is added.) The enzyme cleaves a portion of the enzyme substrate, causing the substrate to form a color, to undergo a color change, to chemiluminesce, or to fluoresce, or causing some other readily detectable phenomenon. In one embodiment, the various elements of the assay, including the solid phase-bound first antibody, the labeled second antibody, and the enzyme or enzyme substrate, are furnished in a single kit used to demonstrate the presence or absence of sperm in a biological or forensic sample.

In one embodiment, a first sperm-specific antibody, preferably a monoclonal antibody, is contacted with a biological or forensic sample under conditions (e.g., aqueous sample, ambient temperature, and normal atmosphere) which permit the antibody-sperm antigen binding reaction to occur. After sufficient reaction time has passed, to the preparation is added a second sperm-specific antibody, which may or may not bind to the same sperm epitope as the first antibody. The second antibody, bearing a reporter molecule or a label such as an enzyme or enzyme substrate, is allowed to bind to sperm bound by the first antibody. In a particular embodiment, the label conjugated to the second antibody is an enzyme. Any unbound or non-specifically bound material, including the second antibody, is removed, e.g., by pouring or washing off the sample. In one embodiment, the first antibody is bound to a solid surface to make the assay simpler and more “user friendly.” A substrate which forms a color, changes color, chemiluminesces, fluoresces, or undergoes some other readily detectable change in the presence of the enzyme, due to the action of the enzyme on the substrate, is then added. Representative enzyme immunosorbent assays (EIA) are described in U.S. Pat. No. 5,149,622, which is incorporated by reference herein in its entirety. Other solid- and liquid-phase assay methodologies may be employed without the exercise of inventive skill.

One embodiment of the invention follows the capture assay format, in which a sperm-specific monoclonal antibody is bound to a solid phase and used to capture the corresponding sperm-specific antigen. Recognition of the sperm-specific antigen may be completed by the use of a second sperm-specific monoclonal or polyclonal immunoreagent coupled to a reporter enzyme, or a third immunoreagent may be employed in a sandwich, as described in Shen et al., 1993, Am. J. Reprod. Immunology 29:231-240.

Another type of assay utilizes a wick (dip stick) and colored beads coated w/a first sperm-specific antibody. A drop of a sperm-containing sample is applied to the antibody-coated colored beads and the beads bound by sperm migrate through a wick until they are captured by a second sperm-specific antibody, which may or may not bind to the same sperm epitope as the first antibody.

Yet another kind of assay employs colored magnetic beads coated w/a first sperm-specific antibody, which may be monoclonal or polyclonal. After mixing the beads with a sample, any sperm cells present are captured by the antibody-coated beads. A magnetic dipstick may be used to recover the magnetic beads. The magnetic source is then deactivated to release the colored magnetic beads. The beads are then allowed to migrate in a wick to a zone containing a second sperm-specific antibody, which captures the sperm-bound beads, resulting in a colored line.

In another assay format, a wick is coated with a first monoclonal antibody specific for a sperm-specific antigen located on or internal to the plasma membrane, and with a second monoclonal antibody specific for a sperm-specific acrosomal antigen such as SP-10. The antibodies may be sprayed onto the wick very close to one another. The first antibody captures any sperm present in a sample. The sperm are then treated to lyse the acrosome. The second antibody captures the acrosomal antigen released after lysis of the acrosome. The wick is then briefly washed. A second sperm acrosome-specific monoclonal or polyclonal antibody conjugated to a reporter molecule or enzyme label may be used to develop a colored reaction product.

In yet another assay format, specialized glass beads with silanized microspikes are employed. The microspikes are coupled to a first antibody specific for a sperm-specific acrosomal antigen such as SP-10. The beads are mixed with a sperm-containing sample to both puncture the acrosome and capture the acrosomal antigen. The beads are then wicked up and detected as a line or spot with a second antibody specific for a sperm-specific acrosomal antigen.

Production of Sperm-Specific Antibodies

Antibodies directed against sperm-specific polypeptides or peptide fragments thereof may be generated using methods that are well known in the art. For instance, U.S. patent application Ser. No. 07/481,491, which is incorporated by reference herein in its entirety, discloses methods of raising antibodies to sperm-specific proteins. For the production of antibodies, various host animals, including but not limited to rabbits, mice, and rats, can be immunized by injection with a sperm-specific polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique which provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for sperm-specific antigens, proteins, derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters. Examples of solid phase peptide synthesis methods include the BOC method which utilized tert-butyloxycarbonyl as the α-amino protecting group, and the FMOC method which utilizes 9-fluorenylmethyloxycarbonyl to protect the α-amino of the amino acid residues, both methods of which are well known by those of skill in the art.

Incorporation of N— and/or C— blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dichloromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide.

Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and decarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for analogs of proteins. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, 10 or more conservative amino acid changes typically have no effect on peptide function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.
   Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The invention also includes a kit comprising the composition of the invention and an instructional material which describes administering the composition to a sample, such as a forensic sample. In another embodiment, this kit comprises a (preferably sterile) solvent suitable for dissolving or suspending the composition of the invention prior to administering the antibody.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the antibodies of the invention or be shipped together with a container which contains the antibody. Alternatively, the instructional material may be shipped separately from the container with the intention that the instructional material and the compound be used cooperatively by the recipient.

Particular Sperm-Specific Antigens

The following description outlines some of the antibodies for use in the invention.

### SP-10 Sperm Protein:

Background on Discovery and Cloning

The testis/sperm-specific, intra-acrosomal sperm antigen SP-10 was identified using MHS-10, a monoclonal antibody (mAb) generated against whole human spermatozoa. SP-10 was identified by immunoblot analysis as a series of protein bands (18-34 kDa), the polymorphism of which was attributed to alternative splicing and endoproteolytic cleavage. Ultrastructural and biochemical studies indicated that SP-10 is a hydrophobic protein localized to the luminal aspect on the inner and outer acrosomal membranes.

Forensic Results

SP-10 appears to remain on the head of sperm recovered from one hour post-coital swabs. The immunofluorescent image is that of a fluorescent cap-shaped organelle. The MHS-10 mAb reacts strongly with sperm heads and not with epithelial cells from the samples.

### ESP Sperm Protein—Sperm Equatorial Segment Protein:

Background on Discovery and Cloning

Two protein spots on a 2-D gel of 36 and 38 kDa and pI 5.1 that reacted with infertile male sera (autoantigenicity) and with ConA (indicating glycosylation) were microsequenced and cloned. The 1.4 Kb cDNA included three in-frame peptides microsequenced from the original spot and hybridized to a single 1.4 Kb testis-specific transcript on a multiple-tissue Northern blot and to testis and placental mRNA on a dot blot of 76 tissues. Computer analysis of the open reading frame (ORF) demonstrated 29% identity and 49% homology over a 68-amino acid C-terminal region (amino acids 278-343) to murine osteoglycin, a secreted extracellular matrix protein. Generation of monospecific rat immune sera allowed localization of the ESP protein to the equatorial segment of human sperm by immunofluorescent and electron microscopy.

Forensic Results

An antibody to ESP reacted with the equatorial segment of sperm heads but not with epithelial cells in samples recovered from one hour post-coital swabs. Considering the frequency with which sperm heads are separated from tails in samples recovered from swabs, antibodies directed against proteins found in sperm heads are particularly useful for sperm immunoselection compositions.

SPAN-X Sperm Protein—Sperm Protein Associated with the X Chromosome:

Background on Discovery and Cloning

SPAN-X is a structural protein associated with the nuclear envelope of spermatozoa. Immunofluorescent labeling demonstrated that SPAN-X is localized to nuclear craters and cytoplasmic droplets of ejaculated human and chimpanzee spermatozoa. Ultrastructurally, the SPAN-X protein is associated with membranous structures within nuclear vacuoles and with the redundant nuclear envelope of human spermatozoa. The ultrastructural localization of the insoluble SPAN-X protein suggests that SPAN-X is a structural component of the sperm nuclear envelope or is associated with structural components of the nucleus, possibly the nuclear matrix. SPAN-X is the first protein specifically localized to these poorly characterized structures of the mammalian sperm nucleus and the first example of a testis-specific protein localized to the nuclear envelope of spermatids.

Significantly, 50% of ejaculated human spermatozoa exhibited immunofluorescent labeling with the SPAN-X antisera. The localization of SPAN-X to 50% of spermatozoa and its X-linked expression by haploid spermatids initially suggested that SPAN-X might be associated with only X-bearing spermatozoa. However, dual labeling of spermatozoa utilizing FISH for the X or Y chromosome and indirect immunofluorescence for the SPAN-X protein demonstrated that SPAN-X is equally distributed between X- and Y-bearing spermatozoa, suggesting that SPAN-X mRNA and/or protein is shared within spermatid cohorts in the testis via cytoplasmic bridges.

Forensic Results

Although SPAN-X is present in only 50% of sperm, the monoclonal antibody A9 generated against recombinant SPAN-X protein would be a valuable component of a cocktail of antibodies for sperm immunoselection. The A9 mAb reacted with sperm heads in samples recovered from post-coital swabs, but it did not react with epithelial cells. Furthermore, this antibody stained SPAN-X localized to the highly convoluted redundant nuclear envelope which lies just beneath the plasma membrane in the cytoplasmic droplet of the spermatozoa. The plasma membrane in this region is easily disrupted, thus exposing the SPAN-X protein and allowing the A9 mAb to bind to SPAN-X.

### CABYR Sperm Protein—Calcium Binding Tyrosine Phosphorylation Regulated Fibrous Sheath Protein Involved in Capacitation:

Background on Discovery and Cloning

CABYR was identified as acidic (pI 4.0) 86 kDa isoforms of a novel, polymorphic, testis-specific protein that were tyrosine-phosphorylated during in vitro capacitation and that bound calcium⁴⁵ on 2-D gels. CABYR is the first demonstration of a sperm protein that gains calcium-binding capacity when phosphorylated during capacitation. Recombinant CABYR has been produced and used to immunize rats to produce polyclonal antisera. With the use of these sera for immunofluorescent and immuno-electron microscopy, CABYR was localized to the principal piece of the human sperm flagellum in association with the fibrous sheath

Forensic Results

Immunofluorescent staining of samples recovered from post-coital swabs using an antibody to CABYR definitively identified sperm tails. This testis-specific protein offers an excellent target for detecting sperm tails.

### SAMP32 Sperm Protein—A Testis-Specific, Isoantigenic, Acrosomal Membrane-Associated Protein:

Background on Discovery and Cloning

SAMP32 was identified in 2-D gel Western blots of sperm extracts containing hydrophobic proteins that partitioned into Triton X-114. Four protein spots with pIs ranging from 4.5 to 5.5 and apparent molecular weights from 32 to 34 kDa were sequenced by mass spectrometry and found to contain common peptide sequences. Cloning the corresponding cDNA revealed that these protein spots were products of a single gene (SAMP32) encoding a protein of 32 kDa with a predicted pI of 4.57. SAMP32 has a potential transmembrane domain in the carboxyl terminus and is phosphorylated in vivo on serine 256. Northern blotting of eight human tissues and RNA dot blotting of 76 human tissues showed that SAMP32 expression was testis-specific. A recombinant form of SAMP32 was produced in E. coli and rat polyclonal sera were produced to this recombinant SAMP32. The antisera strongly stained the equatorial segment and faintly stained the acrosomal cap of ejaculated human spermatozoa by immunofluorescence. Immunoelectron microscopy showed that SAMP32 was associated with the inner acrosomal membrane in the principal and equatorial segments of the sperm acrosome.

Forensic Results

The rat polyclonal antibodies to SAMP32 reacted strongly with sperm heads from post-coital samples. Sperm were identified by a cap- or bar-shaped pattern of immunofluorescence. A low level of reactivity was observed with epithelial cells, which likely can be eliminated by using a higher dilution of the polyclonal antibodies or a monoclonal antibody when one is available.

One of ordinary skill in the art would appreciate that sperm proteins useful in the invention are not limited to the aforementioned proteins or to the antibodies described herein.

### EXAMPLE 1

### Protocol for Staining Sperm Containing Forensic Samples

1. Samples collected on cotton-tipped swabs are rehydrated in 0.5 ml PBS per swab for 20 minutes, agitated manually at 5 minute intervals to release the sample into PBS.
25 μl of sample is applied to a microscope slide and air-dried at room temperature.
2. The sample on the slide is rehydrated with PBS for 5 minutes, the PBS is aspirated,
the sample is fixed with 4% paraformaldehyde for 20 minutes and then washed two times with PBS.
3. The sample is blocked with 10% normal goat serum in PBS for 30 minutes at room temperature.
4. The blocking solution is aspirated and a monoclonal antibody conjugated to a fluorophore is applied to the sample at a concentration of 10 μg/ml in PBS and incubated for 2 hours at room temperature in a humidified chamber.
5. The antibody solution is aspirated and the sample is washed five times with PBS.
6. An antifade reagent is applied and the sample is covered with a coverslip and sealed with nail polish. The slide is stored flat in a covered folder and stored at 4° C.

Directly labeled monoclonal antibodies (single-step reagents) gave very satisfactory results in tests. FIG. 1 and FIG. 2 illustrate the intensity and specificity of such conjugated monoclonal antibodies. The figures represent studies where very few sperm were present in a given field and some sperm heads and tails were separated. The ability of the antibodies to identify sperm heads and tails is highlighted in these figures. AlexaFluor 488 fluorescent dye from Molecular Probes was used to label the monoclonal antibodies. This dye has absorption and emission wavelengths of 494 nm and 519 nm, respectively. The wavelengths can be observed with filters commonly used to observe FITC fluorophores. The fluorophore-to-protein ratio of the conjugates was two compared to the optimal ratio of four to nine. Sperm collected on swabs up to 72 hours after sexual intercourse have also been examined. Preliminary results indicated that sperm still retained the target proteins for these antibodies at this time point.

### EXAMPLE 2

### Loss of Antigens Associated with the Plasma Membrane of Sperm

Following a Human Investigation Committee-approved protocol (HIC #9297), post-coital swabs were collected from 39 volunteer couples. After informed consent was obtained from volunteer couples, they were given sample collection kits containing cotton swabs and labeled boxes with holes that allowed the swabs to air dry. The same boxes are used in sexual assault evidence kits in Virginia hospitals. From each volunteer couple, 10 vaginal swabs were collected at each of four time points ranging between 1 hour and 72 hours after consensual sexual intercourse. Samples were initially investigated at the 2, 6, 12, and 24 hour time points. In some cases swabs were collected at 1, 12, 24, and 72 hours after intercourse. Buccal swabs were also collected from male and female partners to provide control DNA.

Swabs were stored in coolers with ice blocks until they were brought to the study coordinator. They were then stored at 4° C. with desiccant to insure uniformity and prevent bacterial growth. The samples were stained with the S19 monoclonal antibody (described in U.S. Pat. No. 5,830,472, which is incorporated by reference herein in its entirety), which binds to the sperm surface antigen SAGA-1.

Electron Microscopy Revealed Loss of the Plasma Membrane from Sperm Collected Using Current Forensic Techniques Employing Cotton Swabs.

Immunofluorescent experiments indicated that the S19 mAb bound intensely to freshly ejaculated sperm but bound variably and irregularly to sperm eluted from post-coital swabs. This suggested that the SAGA-1 antigen might be lost during the collection, storage, and handling processes of forensic swabs. The loss of the SAGA-1 antigen at some step in the processing might be specific to this antigen or might represent a general loss of the sperm plasma membrane. To test the latter hypothesis, the fine structure of the sperm plasma membrane and the overall morphology of the three following groups were examined as follows: (1) ejaculated sperm that received no additional treatment prior to embedding for electron microscopy; (2) ejaculated sperm that were air-dried onto swabs, stored for six days at room temperature, and recovered prior to embedding; and (3) sperm eluted from post-coital swabs collected one or two hours after intercourse.

Results indicated that the majority of fresh sperm had an intact plasma membrane as well as intact inner and outer acrosomal membranes. However, air-drying fresh sperm had the effect of disrupting the plasma membrane and the acrosome compartment while apparently not affecting the nuclear contents. Post-coital sperm recovered from swabs were completely stripped of the plasma membrane overlying the anterior sperm head and of the outer acrosomal membrane overlying the principal segment of the acrosome. Some sperm eluted from swabs retained the plasma membrane overlying the equatorial segment. Therefore, these results indicated that current methods for the collection, storage, and handling of sexual assault evidence using swabs may not permit the isolation of sperm using reagents directed to a plasma membrane target, such as SAGA-1. Further, it was observed that many of the sperm eluted from swabs had the head detached from the flagellum.

### EXAMPLE 3

### New Sperm-Specific Antigens are Proposed as Targets for Sperm Immunoselection in Forensic Samples

Electron microscopy analysis of sperm recovered from dried swabs indicated that antigens located on the plasma membrane of sperm, such as SAGA-1, may not be the best targets for sperm immunoselection (See Example 2). Consequently, other sperm-specific antigens were considered as potential targets for immunoforensic analysis. Since the sperm head is often separated from the tail in sexual assault evidence recovered from swabs, potential target antigens are sperm head antigens and sperm tail antigens. Table 1 provides a list of some sperm-specific antigens which can be targeted for detecting sperm and for isolating sperm.

TABLE 1

| | |
|---|---|
| ESP | Equatorial Segment Protein, localized to the equatorial |
| | segment of thesperm head. |
| SPAN-X | Major component of the cytoplasmic droplet and localized |
| | to the redundant nuclear membranes and nuclear vacuoles |
| | of 50% of all sperm. |
| CBP86 | Calcium Binding Protein 86, localized to the fibrous |
| | sheath of the principal piece of the sperm tail. |
| SP-10 | Acrosomal matrix protein also associated with acrosomal |
| | membranes. |
| | Some SP-10 remains on the inner acrosomal membrane and |
| | in the equatorial segment after the acrosome reaction. |
| SAMP14 | Sperm Acrosomal Membrane-associated Protein 14, |
| | localized to the acrosome of sperm. |
| SAMP32 | Sperm Acrosomal Membrane-associated Protein 32, |
| | localized to the inner acrosomal membrane and the |
| | equatorial segment of the sperm head. |
| HUP1N | Human sperm protamines 1 and 2, localized to the condensed |
| HUP2B | sperm nucleus. |

Protamines (including protamine 1 and protamine 2), extremely abundant proteins found only in the sperm nucleus, may prove to be effective targets in de-membranated sperm heads recovered from swabs.

Slides of swab smears collected at different time points after sexual intercourse are currently being examined. The slides were prepared by pooling cells eluted from post-coital swabs of three different couples for each time point. Table 2 summarizes preliminary results to date. A greater number of samples, particularly at longer the points where few sperm are present, are being examined to determine the extent to which each antigen persists in sperm in the vagina after intercourse.

TABLE 2

| | 1 Hour | 6 Hours | 12 Hours | 24 Hours | 72 Hours |
|---|---|---|---|---|---|
| ESP | + | + | − | ND | ND |
| mAb 3C6 | | | | | |
| SPAN-X | + | | | | |
| mAb A9 | | | | | |
| CABYR | + | | | | |
| Rat polyclonal | | | | | |
| SP-10 | + | | | | |
| mAb MHS10 | | | | | |
| SAMP32 | + | | | | |
| AKAP3 | + | | + | | |
| Rat polyclonal | | | | | |

(+) denotes positive immunostaining observed; (−) denotes no immunostaining observed; (ND) denotes no data available yet.

For an antigen to be useful in sperm immunoselection in forensic analysis, it must be present and accessible on or in sperm recovered from dried swabs of forensic evidence. Moreover, an antibody to the sperm antigen must not react with other cell types, including vaginal epithelial cells, present in the evidence. A number of anti-sperm antibodies have been tested by immunofluorescent staining of cells recovered from one hour post-coital swabs to confirm that the antibodies would react specifically with sperm and not with epithelial cells present in the samples. Six different antibodies directed against SP-10, CABYR, ESP, SAMP14, SAMP32, and SPAN-X passed this initial screen.

### EXAMPLE 4

### Development of a SpermCheck Forensics Device

Currently undergoing clinical trials are prototypes of SpermCheck Vasectomy, which has been engineered to give a positive signal in post-vasectomy samples with more than 100,000 sperm/ml. Much more sensitive prototypes of the device that can detect as little as 10,000 sperm/ml have also been made.

Although devices designed specifically for forensic samples have not yet been made, Table 3 summarizes the results of a very limited trial with some available prototypes designed to detect the higher sperm concentration limits of SpermCheck Vasectomy. Post-coital samples were collected on cotton swabs from three couples at 1, 6, 12, and 24 hours after intercourse. The dried swabs were stored at 4° C. for more than one year. Each swab was rehydrated in 0.5 ml 10 mM phosphate, 2% Triton X-100, pH 7.2 and agitated to suspend any material extracted from the swab. For each test 140 μl of swab extract was added to the sample well of a device and the result was read after five minutes. Even the SpermCheck Vasectomy prototypes designed for a higher sperm concentration detection limit were able to detect sperm in samples collected 1 and 6 hours after intercourse.

TABLE 3

| Couple | 1 hour | 6 hours | 12 hours | 24 hours |
|---|---|---|---|---|
| #50 | Strong | Negative | Negative | Negative |
| | positive | | | |
| #29 | Strong | Positive | Negative | Negative |
| | positive | | | |
| #24 | positive | Positive | Barely | Negative |
| | | | perceptible | |

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of detecting sperm in a test sample, said method comprising:
obtaining a test sample;
contacting said test sample with a composition comprising at least one antibody directed against a sperm-specific antigen, wherein said at least one antibody is labeled with a reporter molecule; and
detecting said at least one antibody with an assay which measures said reporter molecule, wherein detection of said reporter molecule indicates the presence of sperm in said test sample.

2. The method of claim 1, wherein at least two different antibodies are used.

3. The method of claim 2, wherein at least one of said two different antibodies is directed against a sperm surface antigen.

4. The method of claim 3, wherein said antibody is directed against a sperm head antigen.

5. The method of claim 2, wherein at least one of said antibodies is directed against a sperm-specific nuclear antigen.

6. The method of claim 1, wherein said reporter is measured using an assay selected from a group consisting of a colorimetric assay, a chemiluminescence assay, and a fluorescence assay.

7. The method of claim 2, wherein each of the at least two different antibodies is labeled with different reporter molecules.

8. The method of claim 2, wherein each of the at least two different antibodies is directed against different sperm-specific antigens.

9. The method of claim 1, wherein said sperm-specific antigen is selected from the group consisting of SEQ ID NO:1 (SP-10), SEQ ID NO:2 (CABYR), SEQ ID NO:3 (ESP), SEQ ID NO:4 (SAMP32), SEQ ID NO:5 (SPAN-X), SEQ ID NO:6 (AKAP), CBP86, SAMP-14, HUP1N, and HUP2B.

10. The method of claim 1, wherein said at least one antibody is selected from the group consisting of 3C6, 3A4, 3A5, A9, MHS-10, and 8G8G8G8.

11. The method of claim 1, wherein said test sample is a post-coital swab.

12. A composition for detecting sperm in a sample, said composition comprising at least one antibody directed against a sperm-specific antigen, wherein said antibody is labeled with a reporter molecule.

13. The composition of claim 12, said composition comprising at least two antibodies directed against a sperm-specific antigen, wherein each of the at least two antibodies is directed against different sperm-specific antigens.

14. The composition of claim 12, wherein said sperm-specific antigen is selected from the group consisting of SEQ ID NO:1 (SP-10), SEQ ID NO:2 (CABYR), SEQ ID NO:3 (ESP), SEQ ID NO:4 (SAMP32), SEQ ID NO:5 (SPAN-X), SEQ ID NO:6 (AKAP), CBP86, SAMP-14, HUP1N, and HUP2B.

15. The composition of claim 12, wherein said at least one antibody is selected from the group of antibodies consisting of 3C6, 3A4, 3A5, A9, MHS-10, and 8G8G8G8.

16. A method of purifying sperm DNA from a test sample, said method comprising:
obtaining a test sample;
contacting said test sample with a composition comprising at least one antibody directed against a sperm-specific antigen, wherein said antibody is linked to a support;
allowing whole sperm or sperm nuclei to bind to said at least one antibody;
washing away material which does not bind to the antibody or is non-specifically bound to the antibody;
contacting the bound material with a lysis buffer;
recovering said sperm DNA.

17. The method of claim 16, wherein said recovered sperm DNA is amplified by polymerase chain reaction.

18. The method of claim 16, wherein at least two antibodies are used.

19. The method of claim 16, wherein each of the at least one antibodies is directly linked to the support.

20. The method of claim 16, wherein the support is selected from the group consisting of chromatographic media and magnetic particles.

21. The method of claim 16, wherein said method is automated.

22. The method of claim 16, wherein said test sample is at least 24 hours old.

23. The method of claim 22, wherein said test sample is at least 72 hours old.

24. The method of claim 16, wherein said at least one antibody is labeled with a reporter molecule.

25. The method of claim 16, wherein said at least one antibody is selected from the group consisting of 3C6, 3A4, 3A5, A9, MHS-10, and 8G8G8G8.

26. The method of claim 16, wherein said sperm-specific antigen is selected from the group consisting of SEQ ID NO:1 (SP-10), SEQ ID NO:2 (CABYR), SEQ ID NO:3 (ESP), SEQ ID NO:4 (SAMP32), SEQ ID NO:5 (SPAN-X), SEQ ID NO:6 (AKAP), CBP86, SAMP-14, HUP1N, and HUP2B.

27. The method of claim 16, wherein said test sample is a forensic sample.

28. The method of claim 27, wherein said forensic sample is a post-coital swab.


SEQUENCE LISTING (6 sequences)

SEQ ID NO: 1 - protein, 285 aa, Homo sapiens [sequence omitted]

SEQ ID NO: 2 - protein, 697 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEVTSIVSDNTGQEESGENSVPQEMEGRPVLSGEAAEAVHSGTSVKSSSGPFPPAPEGLTAPEIEPEGESTAEGLMKPAMATSERGQPPPCSNMWTLYCLTDKNQQGHPSPPPAPGPFPQATLYLPNPKDPQFQQHPPKVTFPTYVMGDTKKTSAPPFILVGSNVQEAQGWKPLPGHAVVSQSDVLRYVAMQVPIAVPADEKYQKHTLSPQNANPPSGQDVPRPKSPVFLSVAFPVEDVAKKSSDSGDKCAPFGSYGIAGEVTVTTAHKRRKAETEN

SEQ ID NO: 3 - protein, 350 aa, Homo sapiens [sequence omitted]

SEQ ID NO: 4 - protein, 294 aa, Homo sapiens
MSPRGTGCSAGLLMTVGWLLLAGLQSARGTNVTAAVQDAGLAHEGEGEEETENNDSETAENYAPPETEDVSNRNVVKEVEFGMCTVTCGIGVREVILTNGCPGGESKCVVRVEECRGPTDCGWGKPISESLESVRLACIHTSPLNRFKYMWKLLRQDQQSIILVNDSAILEVRKESHPLAFECDTLDNNEIVATIKFTVYTSSELQMRRSSLPATDAALIFVLTIGVIICVFIIFLLIFIIINWAAVKAFWGAKASTPEVQSEQSSVRYKDSTSLDQLPTEMPGEDDALSEWNE

SEQ ID NO: 5 - protein, 97 aa, Homo sapiens
MDKQSSAGGVKRSVPCDSNEANEMMPETPTGDSDPQPAPKKMKTSESSTILVVRYRRNFKRTSPEELLNDHARENRINPLQMEEEEFMEIMVEIPAK

SEQ ID NO: 6 - protein, 853 aa, Homo sapiens [sequence omitted]